(19) United States
(12) Patent Application Publication
(10) Pub. No.: US 2022/0249430 A1
(43) Pub. Date: Aug. 11, 2022

(54) CONTINUOUS CRYSTALLIZATION OF CANNABINOIDS IN A TUBULAR FLOW REACTOR

(71) Applicant: Canopy Growth Corporation, Smiths Falls (CA)

(72) Inventors: Christopher ADAIR, Smiths Falls (CA); Ben GEILING, Smiths Falls (CA); Scott MCDOUGALL, Smiths Falls (CA); Torbjorn VAN DER MEULEN, Smiths Falls (CA)

(21) Appl. No.: 17/628,863

(22) PCT Filed: Jul. 21, 2020
(86) PCT No.: PCT/CA2020/051008

§ 371 Date: Jan. 20, 2022

Related U.S. Application Data
(60) Provisional application No. 62/877,050, filed on Jul. 22, 2019.

(51) Int. Cl. A61K 31/352; A61K 31/05; A61K 9/16

(52) U.S. Cl. CPC A61K 31/352; A61K 9/1688; A61K 31/05

(57) ABSTRACT

Disclosed herein is a method for producing crystalline cannabinoid particles in continuous mode. The method comprises preparing a cannabinoid-rich solution that comprises a first cannabinoid, and inducing the cannabinoid-rich solution to a supersaturated state in which the first cannabinoid has a supersaturated concentration that is greater than a corresponding saturation concentration of the first cannabinoid. The method further comprises flowing the cannabinoid-rich solution through a tubular reactor in a continuous manner under turbulent flow conditions to form a plurality of crystalline cannabinoid particles and a cannabinoid-depleted solution within the tubular reactor, and separating crystalline cannabinoid particles from the plurality of crystalline cannabinoid particles and the cannabinoid-depleted solution. The turbulent flow conditions are defined by a Reynold number that is greater than a critical Reynolds number for the cannabinoid-rich solution and the tubular reactor.

### CROSS-REFERENCE TO RELATED APPLICATION

This application claims priority to and benefit of U.S. Provisional Patent Application Ser. No. 62/877,050 filed on Jul. 22, 2019, which is hereby incorporated by reference.

### TECHNICAL FIELD

The present disclosure generally relates to methods of crystallizing cannabinoids. In particular, the present disclosure relates to continuous-mode cannabinoid-crystallization methods as opposed to batch-mode cannabinoid-crystallization methods.

### BACKGROUND

Cannabinoids are often defined in pharmacological terms as a class of compounds that exceed threshold-binding affinities for specific receptors found in central-nervous-system tissues and/or peripheral tissues. The interactions between cannabinoids and their receptors are under active investigation by a number of researchers, because the resultant effects are demonstrably important both in medicinal and reactional contexts. Many medicinal and recreational cannabinoid products feature cannabinoids in crystalline form. Methods for producing such products typically rely on batch-mode crystallization parameters. Unfortunately, batch-mode crystallization methods are difficult to control—especially at large scale—and they are often associated with inconsistent product specifications (such as crystal-size distribution, polymorphic form, and/or crystal morphology). Such inconsistent product specifications may negatively impact downstream processes and/or consumer experiences in both medicinal and recreational contexts.

In recent years, continuous crystallization technologies have emerged as viable alternatives to batch-mode crystallization technologies. Continuous-crystallization methods are associated with efficient use of solvents, energy, and space, and they are associated with minimal waste production. Continuous-crystallization methods are designed towards providing near plug-flow conditions (i.e. conditions under which a fluid flows with minimal shearing between adjacent layers). Continuous oscillatory baffled crystallizers (COBCs) are a type of tubular reactor that can be configured to provide near plug-flow conditions in a manner that is suitable for inducing continuous crystallization. COBCs typically feature periodically spaced orifice baffles that oscillate within the tubular reactor (or that remain stationary relative to an oscillated fluid flow) to superimpose oscillatory fluid motion on the net flow. Fluid turbulence in a COBC is associated with the cyclic rise and fall of currents swirling against the primary direction of fluid travel due to fluid-baffle interactions. The fluid mechanics associated with such conditions are highly complex in that there are a number of confounding variables contributing to instantaneous flow conditions (e.g. baffle spacing, baffle geometry, baffle orientation, stroke length, tube diameter, fluid viscosity, etc.). At the same time, there are number of confounding variables that impact the pre-crystallization behavior of a solution as it transitions from an undersaturated state to a supersaturated state and during crystal nucleation/growth. Accordingly, it is difficult to ascertain the particular physical-chemical parameters and flow conditions that will navigate the meta-stable states required to induce continuous crystallization of a particular type of material from a particular solution. Researchers are often confounded when applying systematic approaches to crystallizing target compounds from tubular flow reactors, and this is particularly true for complex mixtures of structurally similar compounds (e.g. regioisomers, stereoisomers, etc.) such as those associated with cannabinoid extracts, resins, distillates, crude isolates, and the like. Accordingly, methods for crystallizing cannabinoids under continuous flow conditions to provide particular crystal-size distributions, polymorphic forms, and/or crystal morphologies are desirable.

### SUMMARY

In contrast to the batch-mode crystallization methods typically employed for cannabinoid crystallization, the methods of the present disclosure utilize tubular flow reactors that are configured for continuous flow under carefully controlled conditions. By modulating the fluid-mechanical parameters of such reactors to exploit the particular solution-phase characteristics of cannabinoids and/or mixtures of cannabinoids, the methods of the present disclosure provide access to a plurality of crystalline cannabinoid materials. In particular, the methods of the present disclosure provide access to crystalline cannabinoid materials with narrow size distributions, and/or narrow polymorphic profiles.

In select embodiments, the present disclosure relates to a method for producing crystalline cannabinoid particles in continuous mode, the method comprising: preparing a cannabinoid-rich solution that comprises a first cannabinoid; inducing the cannabinoid-rich solution to a supersaturated state in which the first cannabinoid has a supersaturated concentration that greater than a corresponding saturation concentration of the first cannabinoid; flowing the cannabinoid-rich solution through a tubular reactor in a continuous manner under turbulent flow conditions to form a plurality of crystalline cannabinoid particles and a cannabinoid-depleted solution within the tubular reactor and to provide a net flow rate through the tubular reactor; and separating crystalline cannabinoid particles from the plurality of crystalline cannabinoid particles and the cannabinoid-depleted solution, or a combination thereof, wherein the turbulent flow conditions are defined by a Reynold number that is greater than a critical Reynolds number for the cannabinoid-rich solution and the tubular reactor.

In select embodiments of the present disclosure, the critical Reynolds number is greater than 2,300. In select embodiments of the present disclosure, the critical Reynolds number is greater than 2,900. In select embodiments of the present disclosure, the critical Reynolds number is greater than 3,900.

In select embodiments of the present disclosure, the Reynolds number is about 6,000.

In select embodiments of the present disclosure, the net flow rate is between about 10 mL/min and about 100 mL/min.

In select embodiments, the methods of the present disclosure further comprise superimposing an oscillating flow rate on the net flow rate by oscillating a piston that is in fluid communication with the tubular reactor.

In select embodiments of the present disclosure, the tubular reactor comprises a baffle that is shaped, oriented, or positioned to partially obstruct flow through the tubular reactor. In select embodiments of the present disclosure, the baffle is one of a plurality of baffles.

In select embodiments, the methods of the present disclosure further comprise oscillating the baffle within the tubular reactor to superimpose an oscillating flow rate on top of the net flow rate.

In select embodiments, the methods of the present disclosure further comprise cooling the cannabinoid-rich solution, the cannabinoid-depleted solution, or a combination thereof within the tubular reactor using a plurality of cooling jackets set to progressively lower temperatures along the tubular reactor.

In select embodiments of the present disclosure, the first cannabinoid is THC (Δ9-THC), THCA, Δ8-THC, trans-Δ10-THC, cis-Δ10-THC, THCV, THCVA, Δ8-THCV, Δ9-THCV, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA CBL, CBLA, CBLV, CBLVA, CBT, CBTA, or cannabicitran.

In select embodiments of the present disclosure, the cannabinoid-rich solution comprises a cannabinoid extract, a cannabinoid resin, a cannabinoid distillate, a cannabinoid isolate, or a combination thereof.

In select embodiments of the present disclosure, the cannabinoid-rich solution comprises THC (Δ9-THC), THCA, Δ8-THC, trans-Δ10-THC, cis-Δ10-THC, THCV, THCVA, Δ8-THCV, Δ9-THCV, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA CBL, CBLA, CBLV, CBLVA, CBT, CBTA, cannabicitran, or a combination thereof.

In select embodiments of the present disclosure, the cannabinoid-rich solution comprises a solvent. The solvent may comprise pentane, hexane, heptane, methanol, ethanol, isopropanol, dimethyl sulfoxide, acetone, ethyl acetate, diethyl ether, tert-butyl methyl ether, water, acetic acid, anisole, 1-butanol, 2-butanol, butane, butyl acetate, ethyl formate, formic acid, isobutyl acetate, isopropyl acetate, methyl acetate, 3-methyl-1-butanol, methylethyl ketone, 2-methyl-1-propanol, 1-pentanol, 1-propanol, propane, propyl acetate, trimethylamine, or a combination thereof. In select embodiments, the solvent is heptane.

In select embodiments of the present disclosure, the cannabinoid-rich solution has a viscosity of between about 0.05 cP and about 250 cP at an inlet to the tubular reactor.

In select embodiments of the present disclosure, the cannabinoid-rich solution has a fluid density of between about 0.2 g/mL and about 1,700 g/mL at an inlet to the tubular reactor.

In select embodiments of the present disclosure, the cannabinoid-rich solution has a temperature of between about 0° C. and about 50° C. at an inlet to the tubular reactor.

In select embodiments of the present disclosure, the inducing of the cannabinoid-rich solution to the supersaturated state precedes the flowing of the cannabinoid-rich solution through the tubular reactor.

In select embodiments of the present disclosure, the inducing of the cannabinoid-rich solution to the supersaturated state is concurrent the flowing of the cannabinoid-rich solution through the tubular reactor.

In select embodiments, the methods of the present disclosure further comprise dispersing a plurality of seed crystals into the cannabinoid-rich solution concurrent with the flowing of the cannabinoid-rich solution through the tubular reactor.

Other aspects and features of the methods of the present disclosure will become apparent to those ordinarily skilled in the art upon review of the following description of specific embodiments.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1A shows a schematic diagram of an exemplary tubular reactor in accordance with the present disclosure.

FIG. 1B shows a schematic diagram of an exemplary tubular reactor during forward movement of an oscillating piston.

FIG. 1C shows a schematic diagram of an exemplary tubular reactor during backward movement of an oscillating piston.

FIG. 2A shows a schematic diagram of a segment of an exemplary tubular reactor.

FIG. 2B shows a schematic diagram of a segment of an exemplary tubular reactor 100 comprising baffles under forward flow conditions.

FIG. 2C shows a schematic diagram of a segment of an exemplary tubular reactor 100 comprising baffles under back flow conditions.

FIG. 2D shows a cross-sectional view of a schematic diagram of an exemplary tubular reactor 100 having a baffle 120 that comprises an annular region that obstructs flow surrounding a circular opening 122 that permits flow.

FIG. 2E shows a cross-sectional view of a schematic diagram of an exemplary tubular reactor 100 and an exemplary baffle 120 that defines part of an opening 122.

FIG. 3 shows a flow diagram of a method in accordance with the present disclosure.

FIG. 4 shows an exemplary harvest of crystals obtained from a method in accordance with the present disclosure.

FIG. 5A shows a 10× magnification of crystals obtained from a method in accordance with the present disclosure.

FIG. 5B shows a 10× magnification of crystals obtained from a conventional batch process.

FIG. 6A shows a 40× magnification of crystals obtained from a method in accordance with the present disclosure.

FIG. 6B shows a 40× magnification of crystals obtained from a conventional batch process.

### DETAILED DESCRIPTION

Careful analysis of the solution-phase behavior of a range of cannabinoid solutions indicates that: (i) undersaturated cannabinoid solutions can be induced to supersaturate with strategic manipulation of temperature, pressure, solute, solvent, co-solvent, and/or non-solvent parameters; and (ii) turbulent flow conditions can be used to induce cannabinoid crystallization from supersaturated cannabinoid solution under controlled, repeatable conditions. In the context of the present disclosure the terms “supersaturate” and “supersaturation” refer to a meta-stable state in which a solution comprises a kinetically unstable quantity of solute such that spontaneous nucleation and/or induced nucleation is likely to occur. The methods of the present disclosure leverage the combination of such kinetically unstable solutions with turbulent flow conditions to induce crystal nucleation and growth under controlled, repeatable conditions. The result is a series of methods that provide crystalline cannabinoid materials with narrow crystal-size distributions and consistent polymorphic characteristics. Such crystalline cannabinoids are likely to be useful in both medical and recreational contexts.

In select embodiments, the present disclosure relates to a method for producing crystalline cannabinoid particles in continuous mode, the method comprising: preparing a cannabinoid-rich solution that comprises a first cannabinoid; inducing the cannabinoid-rich solution to a supersaturated state in which the first cannabinoid has a supersaturated concentration that greater than a corresponding saturation concentration of the first cannabinoid; flowing the cannabinoid-rich solution through a tubular reactor in a continuous manner under turbulent flow conditions to form a plurality of crystalline cannabinoid particles and a cannabinoid-depleted solution within the tubular reactor and to provide a net flow rate through the tubular reactor; and separating crystalline cannabinoid particles from the plurality of crystalline cannabinoid particles, the cannabinoid-depleted solution, or a combination thereof, wherein the turbulent flow conditions are defined by a Reynold number that is greater than a critical Reynolds number for the cannabinoid-rich solution and the tubular reactor.

As will be appreciated by those skilled in the art who have benefitted from the teachings of the present disclosure, inducing a cannabinoid-rich solution into a supersaturated state is a prerequisite to cannabinoid crystallization. Such inducing may be driven by a variety of factors such as temperature decrease, pH adjustment, solute addition, solvent evaporation, co-solvent addition, non-solvent addition, or a combination thereof. Those skilled in the art who have benefitted from the teachings of the present disclosure will readily appreciate that the particulars of any such strategy for inducing a cannabinoid-rich solution into a supersaturated state will vary, but that the general concepts are similar such that the following discussion of temperature-driven supersaturation may be extrapolated to alternate approaches. With respect to temperature-driven supersaturation of a cannabinoid-rich solution, the particular cannabinoid-rich solution may be characterized by a solubility curve (i.e. a plot of saturation concentration as a function of temperature for a particular pressure) which delineates a boundary between undersaturation and supersaturation conditions. When a hot and undersaturated cannabinoid-rich solution is cooled, it approaches the corresponding point on the solubility curve (i.e. its saturation point). With further cooling, the cannabinoid-rich solution becomes supersaturated, such that the supersaturated solution is in a metastable state, wherein a suitable initiation event will induce nucleation and crystal growth. Nucleation and crystal growth decrease the concentration of the particular cannabinoid (i.e. desupersaturation), and the cannabinoid-rich solution is depleted towards the corresponding point on the solubility curve. While the solubility of the particular cannabinoid in solution may be readily determined experimentally, the supersolubility or the metastable limit for the cannabinoid-rich solution is difficult to define, because it depends on numerous factors such as the rate of supersaturation generation (i.e. cooling rate). The results disclosed herein indicate that inducing the cannabinoid-rich solution to a supersaturated state in which the particular cannabinoid has a supersaturated concentration that is greater than its corresponding saturation concentration provides sufficient supersaturation for turbulent-flow-induced nucleation and/or growth in a continuous fashion provided the turbulent-flow related parameters meet particular conditions.

As will be appreciated by those skilled in the art who have benefited from the teachings of the present disclosure, turbulent flow conditions within a tubular reactor may be quantified by the oscillatory Reynolds number (Reₒ) as defined as by EQN. 1.

$${Re}_{o} = \frac{2 π {fx}_{o} ρ D}{μ} \qquad ( EQN . 1 )$$

Wherein:

D is the diameter of the tubular reactor (in m);

ρ is the fluid density (in kg/m³);

μ is the fluid viscosity (kg/m·s);

xₒ is the oscillation amplitude (m); and

f is the oscillation frequency.

The oscillatory Reynolds number describes the intensity of mixing within a tubular reactor under oscillating flow conditions such as those utilized in continuous oscillatatory baffled crystallizers (COBCs). Those skilled in the art who have benefited from the teachings of the present disclosure will readily ascertain the related Reynolds number equations for alternate types of tubular reactors. The severity of the turbulent flow conditions required to induce crystal nucleation/growth from a particular cannabinoid-rich solution will vary depending on the particulars of the cannabinoid rich solution and the extent of supersaturation. However, for any particular solution/reactor combination, there is a critical Reynolds number that defines the minimum turbulent flow conditions required to induce crystal nucleation/growth.

In the context of the present disclosure, the terms “crystal”, “crystallizing”, “crystalline”, are used broadly to refer to a spectrum of solid materials having a degree of microscopic order but not necessarily a highly ordered crystal lattice that extends in all directions. As will be appreciated by those skilled in the art who have benefitted from the teachings of the present disclosure, the degree of crystallinity of material can be evaluated by a variety of means such as but not limited to powder X-ray diffraction, single-crystal X-ray diffraction, differential scanning calorimetry, and the like.

The cannabinoid-rich solution may be prepared from a distillate, a resin, an extract, or the like. For example, the cannabinoid-rich solution may be prepared by solvent extraction from marijuana or hemp. The extraction solvent may be supercritical carbon dioxide, ethanol, heptane, pentane, propane, n-butane, iso-butane, or any Class 3 solvent as defined by the International Conference on Harmonization (ICH) guidelines. Optionally, the solvent extract (or the cannabinoid-rich solution more generally) may be further refined using purification processes including but not limited to filtration, winterization (i.e. precipitation of undesired plant waxes using organic solvent at or below ambient temperature), distillation, chromatography (e.g. normal-phase, reversed-phase, centrifugal partition, simulated moving bed), trituration, liquid-liquid extraction, and/or solid-liquid extraction. The cannabinoids in the cannabinoid-rich solution may also be of synthetic origin.

The cannabinoids of the cannabinoid-rich solution may also be combined with an excipient(s) (e.g. basic molecules, acidic molecules, co-formers, derivatization agents) prior to crystallization to modify the physical and/or chemical properties of the cannabinoids and/or the cannabinoid-rich solution. For example, cannabinoids may be incubated with a basic molecule, such as an alkaloid or basic amino acid, to form the carboxylate salt of the cannabinoid of interest. This modification may serve to improve the crystallization process and/or to modify the physical/chemical properties of the cannabinoid of interest. Cannabinoids of the cannabinoid-rich solution may also be combined with acidic molecules and/or co-formers prior to crystallization to form a co-crystal with the cannabinoid of interest during the crystallization process. This modification may serve to improve the crystallization process and/or to modify the physical/chemical properties of the cannabinoid of interest.

Cannabinoids of the cannabinoid-rich solution may also be combined with derivatization agents prior to crystallization to form a chemical derivative of the cannabinoid of interest. This modification may serve to improve the crystallization process and/or to modify the physical/chemical properties of the cannabinoid of interest. For example, tetrahydrocannabinol (THC) is an oil under ambient conditions and does not readily crystallize. Functionalization of the phenolic hydroxyl group of THC with certain ester or sulfonic ester moieties may enable crystallization of otherwise non-crystallizable cannabinoids including but not limited to Δ⁹-tetrahydrocannabinol, Δ⁸-tetrahydrocannabinol, and/or cannabinol. Alternatively, esters of acidic cannabinoids made by synthetic means can be used as the cannabinoids in the cannabinoid cannabinoid-rich solution.

As used herein, the term “cannabinoid” refers to: (i) a chemical compound belonging to a class of secondary compounds commonly found in plants of genus cannabis, (ii) synthetic cannabinoids and any enantiomers thereof; and/or (iii) one of a class of diverse chemical compounds that may act on cannabinoid receptors such as CB1 and CB2.

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In select embodiments of the present disclosure, the cannabinoid is a compound found in a mammal, sometimes called an endocannabinoid.

In select embodiments of the present disclosure, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form). As will be appreciated by those skilled in the art who have benefitted from the teachings of the present disclosure, the terms “first cannabinoid” may refer to: (ii) salts of acid forms, such as Na⁺ or Ca²⁺ salts of such acid forms; and/or (iii) ester forms, such as formed by hydroxyl-group esterification to form traditional esters, sulphonate esters, and/or phosphate esters.

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC), Tetrahydrocannabinolic acid C4 (THCA-C4), Tetrahydrocannabinol C4 (THC-C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11 nor 9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-05), Dehydrocannabifuran (DCBF), Cannbifuran (CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

Within the context of this disclosure, where reference is made to a particular cannabinoid without specifying if it is acidic or neutral, each of the acid and/or decarboxylated forms are contemplated as both single molecules and mixtures.

As used herein, the term “THC” refers to tetrahydrocannabinol. “THC” is used interchangeably herein with “Δ9-THC”.

In select embodiments of the present disclosure, the first cannabinoid may comprise THC (Δ9-THC), THCA, Δ8-THC, trans-Δ10-THC, cis-Δ10-THC, THCV, THCVA, Δ8-THCV, Δ9-THCV, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, CBLVA CBT, CBTA, or cannabicitran.

Structural formulae of cannabinoids of the present disclosure may include the following:

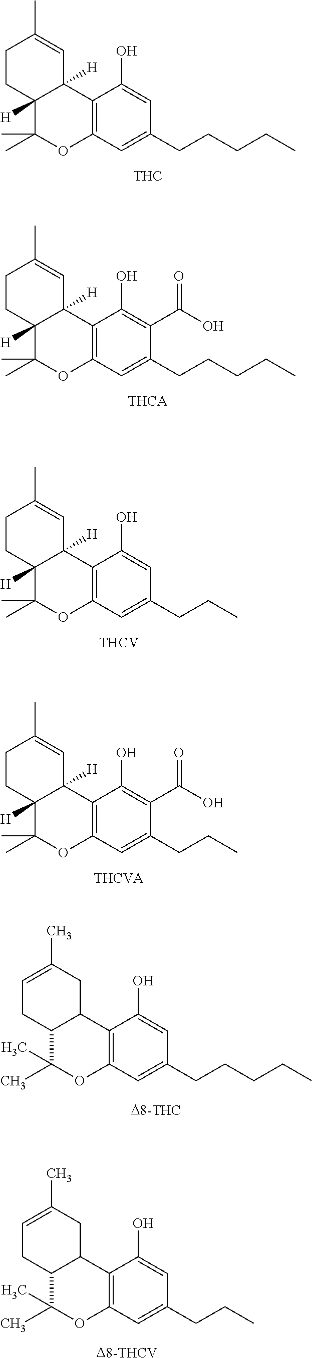

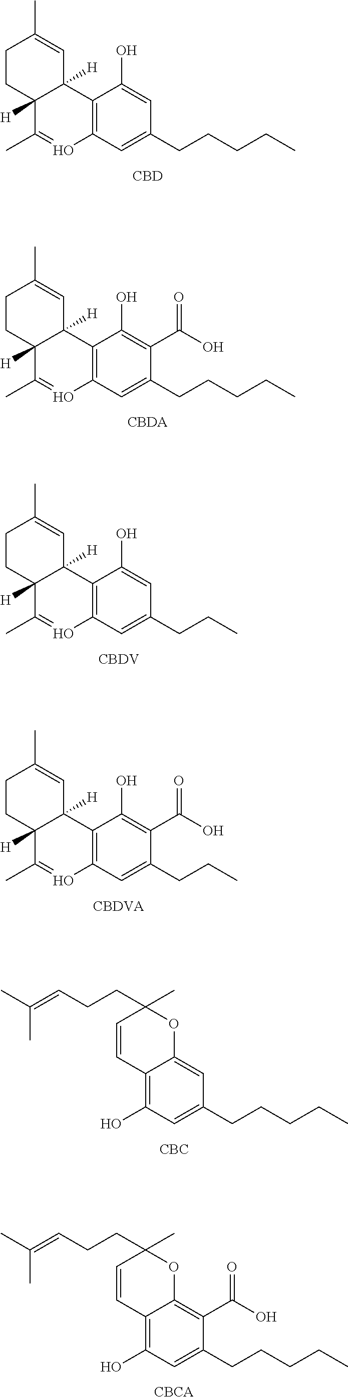

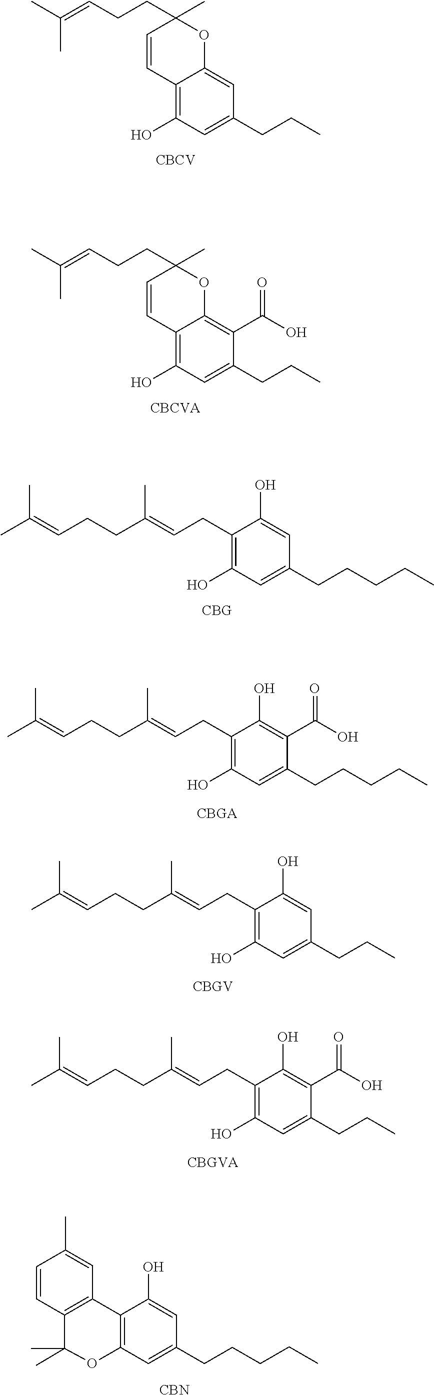

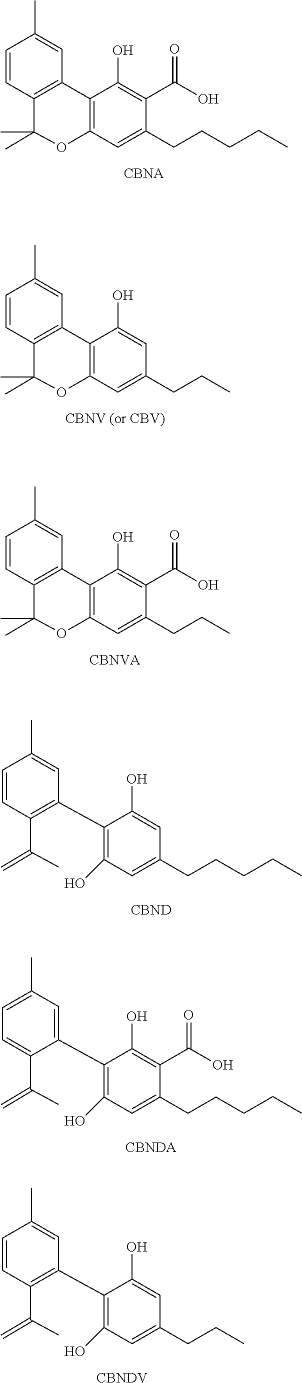

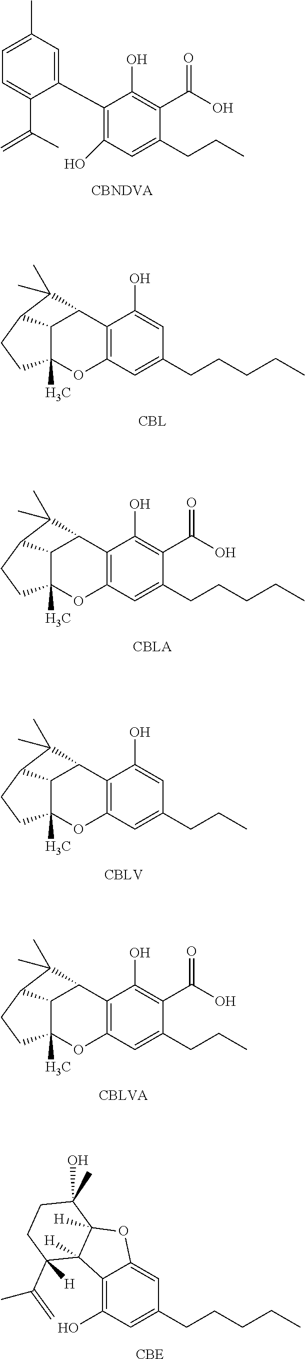

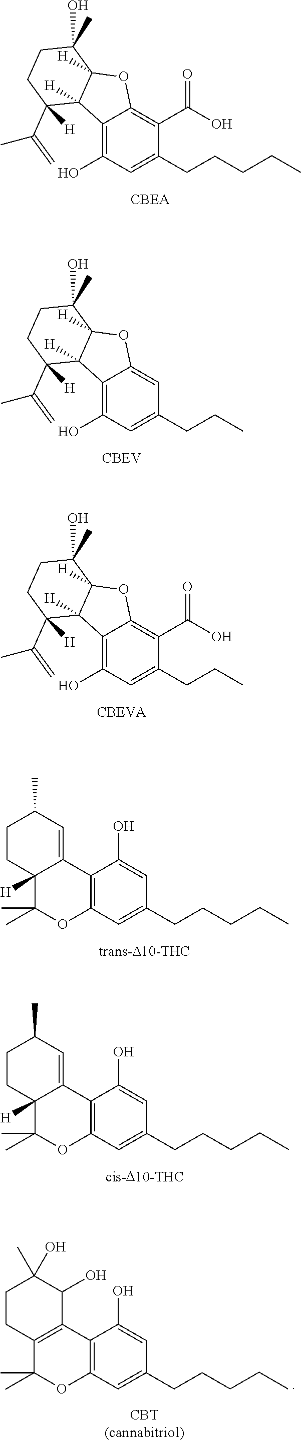

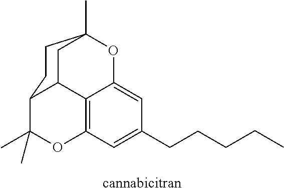

In select embodiments of the present disclosure, the first cannabinoid may comprise CBD, CBDV, CBC, CBCV, CBG, CBGV, THC, THCV, or a regioisomer thereof. As used herein, the term “regioisomers” refers to compounds that differ only in the location of a particular functional group.

In select embodiments of the present disclosure, the first cannabinoid is CBD.

In select embodiments of the present disclosure, the first cannabinoid is Δ⁹-THC or Δ¹⁰-THC.

As noted above, inducing a cannabinoid-rich solution into a supersaturated state is a prerequisite to cannabinoid crystallization. In select embodiments of the present disclosure, the inducing of the cannabinoid-rich solution to the supersaturated state may precede the flowing of the cannabinoid-rich solution through the tubular reactor. Alternatively, the inducing of the cannabinoid-rich solution to the supersaturated state may be concurrent the flowing of the cannabinoid-rich solution through the tubular reactor.

In select embodiments of the present disclosure, the supersaturation of a cannabinoid-rich solution may be induced by controlled temperature reduction. Hence, the methods of the present disclosure may comprise cooling the cannabinoid-rich solution, the cannabinoid-depleted solution, or a combination thereof within the tubular reactor. For example, the cannabinoid-rich solution may comprise cannabidiol (CBD) with a CBD concentration of about 1 g/mL of solvent. In select embodiments of the present disclosure, the cannabinoid-rich solution may have a temperature of between about 0° C. and about 50° C. at an inlet to the tubular reactor.

In select embodiments of the present disclosure, the cannabinoid-rich solution may have a viscosity of between about 0.05 cP and about 250 cP at an inlet to the tubular reactor. For example, the cannabinoid-rich solution may comprise CBD and have a viscosity of about 1 cP.

In select embodiments of the present disclosure, the cannabinoid-rich solution may have a fluid density of between about 0.2 g/mL and about 1,700 g/mL at an inlet to the tubular reactor. For example, the cannabinoid-rich solution may comprise CBD and have a fluid density of about 1.0 g/mL.

As noted above, the severity of the turbulent flow conditions required to induce crystal nucleation/growth from a particular cannabinoid-rich solution will vary depending on the particulars of the cannabinoid rich solution and the extent of supersaturation. However, for any particular solution/reactor combination, there is a critical Reynolds number to induce turbulent flow. In select embodiments of the present disclosure, the critical Reynolds number may be greater than 2,300, 2,900, or 3,900.

As will be appreciated by those skilled in the art who have benefitted from the teachings of the present disclosure, the length and/or cross sectional shape of the tubular reactor can take any of a variety of forms provided that turbulent flow conditions are achieved within the tubular reactor. Likewise, the hydraulic diameter of the tubular reactor is not limited to any particular value and can vary, provided that turbulent flow conditions are achieved within the tubular reactor. For example, turbulent flow conditions for a cannabinoid solution have a fluid density of 1.7 g/mL and a fluid viscosity of 1 cP may be achieved in a tubular reactor that has a diameter of about 5 cm provided the product of the oscillating frequency and amplitude is sufficiently high.

In select embodiments of the present disclosure, the tubular flow reactor may be configured to provide turbulent flow conditions as characterized by a Reynolds number that is above the critical Reynolds number and that is: (i) between about 3,000 and about 5,000, (ii) between about 5,000 and about 7,000, (iii) between about 7,000 and about 9,000, (iv) between about 9,000 and about 11,000, (v) between about 11,000 and about 13,000, and/or (vi) between about 13,000 and about 15,000. For example, the tubular flow reactor may be configured to provide turbulent flow conditions as characterized by a Reynolds number that is about 3000, about 3500, about 4000, about 4500, about 5000, about 5500, about 6000, about 6500, about 7000, about 7500, about 8000, about 8500, about 9000, about 9500, about 10,000, about 10,500, about 11,000, about 11,500, about 12,000, about 12,500, about 13,000, about 13,500, about 14,000, about 14,500 or about 15,000.

In select embodiments of the present disclosure, the tubular flow reactor may be configured to provide turbulent flow conditions as characterized by a Reynolds number that is above the critical Reynolds number and that is: (i) between about 3,000 and about 15,000, (ii) between about 4,000 and about 13,000, and/or (iii) between about 5,000 and about 9,000. For example, the tubular flow reactor may be configured to provide turbulent flow conditions as characterized by a Reynolds number that is about 3000, about 3500, about 4000, about 4500, about 5000, about 5500, about 6000, about 6500, about 7000, about 7500, about 8000, about 8500, about 9000, about 9500, about 10,000, about 10,500, about 11,000, about 11,500, about 12,000, about 12,500, about 13,000, about 13,500, about 14,000, about 14,500 or about 15,000.

In the context of the present disclosure, if the Reynolds number for a given set of reactor conditions is above a critical Reynolds number, the flow is turbulent, and if the Reynolds number is below an alternative critical Reynolds number, the flow is laminar. In select embodiments of the present disclosure, higher flow rates, lower fluid viscosities, and smaller hydraulic diameters for the reactor increase the Reynolds number. In select embodiments of the present disclosure, the presence of obstructions to flow within the reactor may decrease the critical Reynolds number. In select embodiments of the present disclosure, such flow obstructions may comprise baffles.

In select embodiments of the present disclosure, the net flow rate through the tubular reactor may between about 10 mL/min and about 100 mL/min. Those skilled in the art having benefited from the teachings of the present disclosure will appreciate that such flow rates apply to laboratory-scale tubular flow reactors and that larger scale tubular flow reactors are associated with higher flow rates. The flow rate may be characterized as a linear flow rate, wherein the flow-rate is expressed in linear velocity units such as meters per second (m/s). Alternatively, the flow rate may be expressed as a volumetric flow rate. For example, flow rates may be expressed in volumetric velocity units such as liters per hour (L/h). Flow rates may also be expressed as a ratio of the total volume of the tubular reactor divided by the volumetric flow rate, for example the residence time of fluid within the reactor (h). Moreover, an oscillating flow rate may be superimposed on the net flow rate by oscillating a piston that is in fluid communication with the tubular reactor. For example, an oscillating the flow may be executed by reversibly translating the piston at a frequency between about 0.1 Hz and about 6.0 Hz on the laboratory scale. Those skilled in the art who have benefitted from the teachings of the present disclosure will appreciated that larger scale tubular reactors may be suited to alternate piston-oscillation protocols. The tubular reactor may comprise a baffle that is shaped, oriented, and positioned to partially obstruct flow through the tubular reactor. The baffle may be one of a plurality of baffles, and the baffle (or plurality of baffles) may be oscillated within the tubular reactor to superimpose an oscillating flow rate on top of the net flow rate.

In select embodiments of the present disclosure, the cannabinoid-rich solution may comprise a solvent. The solvent may comprise pentane, hexane, heptane, methanol, ethanol, isopropanol, dimethyl sulfoxide, acetone, ethyl acetate, diethyl ether, tert-butyl methyl ether, water, acetic acid, anisole, 1-butanol, 2-butanol, butane, butyl acetate, ethyl formate, formic acid, isobutyl acetate, isopropyl acetate, methyl acetate, 3-methyl-1-butanol, methylethyl ketone, 2-methyl-1-propanol, 1-pentanol, 1-propanol, propane, propyl acetate, trimethylamine, or a combination thereof.

In select embodiments, the methods of the present disclosure further comprise dispersing a plurality of seed crystals into the cannabinoid-rich solution concurrent with the flowing of the cannabinoid-rich solution through the tubular reactor. In the context of the present disclosure, a seed crystal dispersion comprises at least one seed crystal dispersed in at least one solvent. A seed crystal is a crystal with a size smaller than the crystal size of the desired product. Addition of seed crystals may be advantageous as the presence of seed crystals obviates the need for nucleation during crystallization, and so long as the seed crystal size and size distribution remain constant the size and size distribution of the resulting crystalline cannabinoid particles will remain constant. Variations in the rate of nucleation contribute to variation in size distribution, therefore obviating the need for nucleation may increase the reliability of the crystallization.

Crystalline cannabinoids produced by continuous crystallization can be modified by means of salt formation, co-crystallization, derivatization (e.g. esterification, etherification, isomerization, alkylation, arylation, oxidation, reduction, ring-opening/ring-closing reactions). Previously crystallized material can be further purified by recrystallization using COBC to obtain enhanced cannabinoid purity. Recrystallization may also be conducted on modified crystal forms (e.g. carboxylate salts, co-crystals, synthetic derivatives) originally obtained by continuous crystallization.

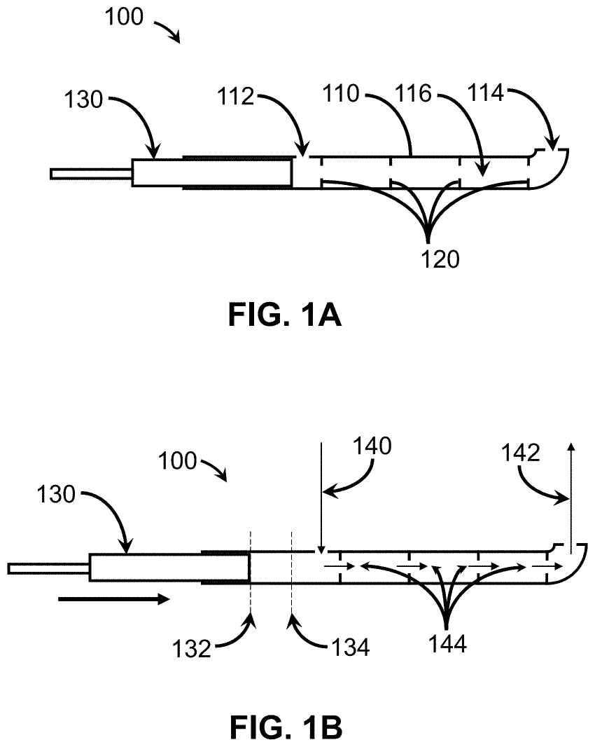

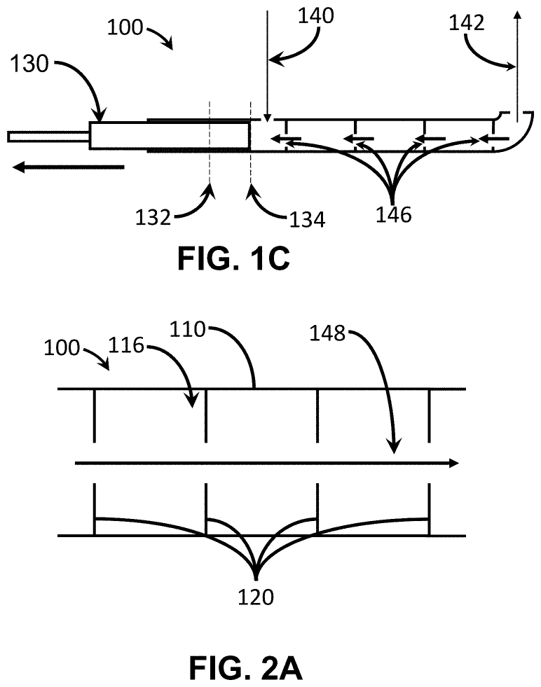

Embodiments of the present disclosure will now be described by reference to FIG. 1 to FIG. 6.

FIG. 1A shows a schematic diagram of an exemplary tubular reactor 100 which may be employed in executing a method in accordance with the present disclosure. Tubular reactor 100 comprises a tubular casing 110, an inlet 112, an outlet 114, a plurality of baffles 120, and a piston 130. The tubular casing 110 defines a tubular volume 116 through which a cannabinoid-rich solution, a cannabinoid-depleted solution, and/or a slurry of crystalline cannabinoid particles may flow. Inlet 112 comprises an opening in tubular casing 110 through which the cannabinoid-rich solution may enter tubular volume 116. Outlet 114 comprises an opening in tubular casing 110 through which the cannabinoid-depleted solution, and/or the slurry of crystalline cannabinoid particles may exit tubular volume 116. In other words, the cannabinoid-rich solution, the cannabinoid-depleted solution, and/or the slurry of crystalline cannabinoid particles may flow through tubular reactor 100 by entering tubular reactor 100 through inlet 112, flowing through tubular volume 116, and exiting tubular reactor 100 through outlet 114. Flowing fluid through tubular reactor 100 may advantageously be performed under turbulent flow conditions as defined by the critical Reynolds number as set out above.

Oscillating piston 130 may move forwards and backwards, causing fluid within tubular volume 116 to oscillate forwards and backwards. FIG. 1B shows a schematic diagram of tubular reactor 100 during forward movement of oscillating piston 130. Fluid enters tubular reactor 100 at input flow rate 140, while fluid exits tubular reactor 100 at output flow rate 142. Averaged over time, input flow rate 140 and output flow rate 142 are equal to one another and are both equal to net flow rate 148. Oscillating piston 130 begins at position 132 and moves forward to position 134. As oscillating piston 130 moves forward, oscillating piston 130 forces fluid within tubular volume 116 forward, generating forward flow 144. Forward flow 144 combines additively with net flow rate 148 to increase the instantaneous flow rate through tubular reactor 100.

FIG. 1C shows a schematic diagram of an exemplary tubular reactor 100 during backward movement of oscillating piston 130. Oscillating piston 130 begins at position 134 and moves forward to position 132. The distance between position 132 and 134 comprises an oscillation amplitude. The rate at which piston 130 oscillates between position 132 and 134 comprises the oscillation frequency. As oscillating piston 130 moves backward, oscillating piston 130 forces fluid within tubular volume 116 backward, generating back flow 146. Back flow 146 combines subtractively with net flow rate 148 to decrease the instantaneous flow rate through tubular reactor 100. If back flow 146 is greater than net flow rate 148, then back flow 146 will cause flow within tubular reactor to reverse. Oscillating between forward flow and back flow allows for relatively high, albeit temporary, flow rates within tubular reactor 100 without increasing the net flow rate within tubular reactor 100. The level of turbulence within tubular reactor 100 increases with increasing oscillation amplitude and frequency, without any need to increase net flow rate 148, thereby increasing the turbulence of flow within tubular flow reactor 100 without decreasing the residence time of fluid within tubular flow reactor 100. In other words, oscillating flow allows the tubular reactor to operate with an oscillatory Reynolds number above the critical oscillatory Reynolds number for turbulent flow without requiring a high net flow rate.

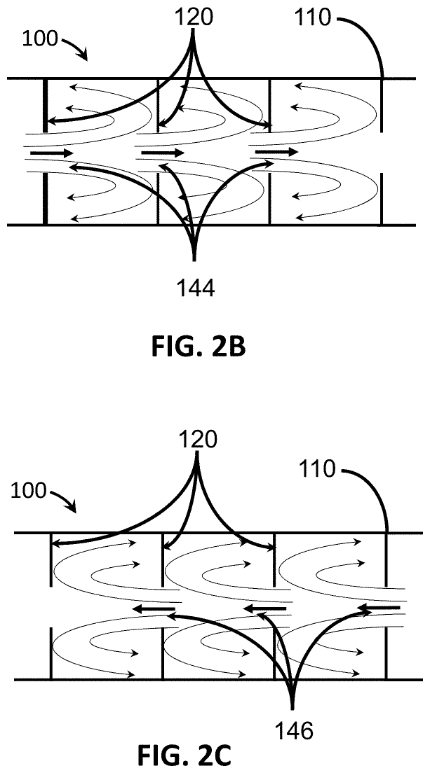

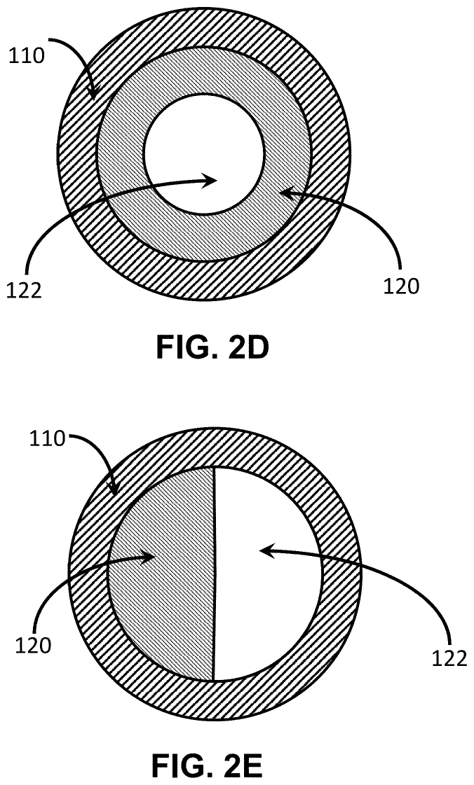

FIG. 2A shows a schematic diagram of a segment of tubular reactor 100. Fluid may flow through tubular reactor 100 at net flow rate 148. Net flow rate 148 comprises the average flow rate of fluid through tubular reactor 100. Tubular reactor 100 may comprise at least one baffle 120. Each baffle 120 at least partially obstructs the flow of fluid through tubular reactor 100. The portion of tubular volume 116 bounded by a baffle 120 and an immediately adjacent baffle 120 may comprise an inter-baffle zone, where tubular volume 116 may be sub-divided into a plurality of inter-baffle zones by a plurality of baffles.

FIG. 2B shows a schematic diagram of a segment of tubular reactor 100 comprising baffles 120 under forward flow conditions. Forward flow 144 must flow around each baffle 120 creating axial flow within tubular reactor 100, wherein axial flow comprises flow from the center of tubular reactor 100 towards tubular casing 110. Axial flow may comprise a vortex.

FIG. 2C shows a schematic diagram of a segment of tubular reactor 100 comprising baffles 120 under back flow conditions. Back flow 146 must flow around each baffle 120 creating axial flow within tubular reactor 100. Each oscillation between forward and back flow reverses not only the direction of flow, but the direction of vortices formed due to axial flow around each baffle 120. Forming, then reversing the direction, of vortices significantly increases the turbulence of flow within tubular reactor 100. In other words, baffles may cause flow conditions within a tubular reactor to be turbulent by lowering the critical Reynolds number for turbulent flow.

FIG. 2D shows a cross-sectional view of baffle 120 within tubular casing 110. Baffle 120 comprises an annular region that obstructs flow surrounding a circular opening 122 that permits flow. Baffle 120 is physically coupled to tubular casing 110. Those skilled in the art who have benefitted from the present disclosure will appreciate that while both tubular casing 110 and baffle 120 are depicted as annular, each of tubular casing 110 and baffle 120 may be of any appropriate cross-section that partially obstructs and partially permits flow through tubular reactor 100. Furthermore, while baffle 120 is depicted in FIG. 2D as completely surrounding opening 122, this is not always the case. For example, FIG. 2E shows a cross-sectional view of an exemplary baffle 120 where opening 122 comprises the space between baffle 120 and tubular casing 110.

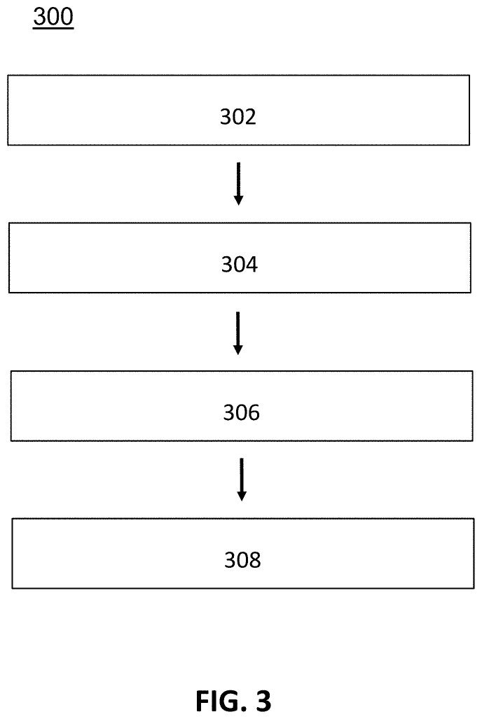

FIG. 3 shows a flow diagram for a method 300 for continuously preparing crystalline cannabinoid particles. Method 300 comprises four steps: preparing a cannabinoid-rich solution that comprises a first cannabinoid (step 302); inducing the cannabinoid-rich solution to a supersaturated state in which the first cannabinoid has a supersaturated concentration that is greater than a corresponding saturation concentration of the first cannabinoid (step 304); flowing the cannabinoid-rich solution through a tubular reactor in a continuous manner under turbulent flow conditions to form a plurality of crystalline cannabinoid particles and a cannabinoid-depleted solution within the tubular reactor and to provide a net flow rate through the tubular reactor (step 306); and separating crystalline cannabinoid particles from the plurality of crystalline cannabinoid particles and the cannabinoid-depleted solution (step 308). In step 304, the turbulent flow conditions are defined by a Reynold number that is greater than a critical Reynolds number for the cannabinoid-rich solution and the tubular reactor. Throughout the description of method 300, related elements from FIGS. 1A, 1B, 1C, 2A, 2B, 2C, 2D, and/or 2E are called out in brackets for exemplary purposes.

In the embodiment illustrated in FIG. 3, steps 304 and 306 are executed as a single step during which a cannabinoid-rich solution is flowed through a tubular reactor (100) under turbulent flow conditions to form a slurry of crystalline cannabinoid particles within the tubular reactor. The turbulent flow conditions are characterized by a flow rate, a fluid viscosity, and a reactor hydraulic diameter which define a Reynolds number that is above a critical Reynolds number for turbulent flow. Concurrent with the flowing through tubular reactor 100, the cannabinoid-rich solution is cooled to a supersaturation state. The slurry of crystalline cannabinoid particles comprises at least one solvent, a reduced amount of the at least one cannabinoid dissolved in the at least one solvent, and crystalline cannabinoid particles. In this context, forming larger crystals may be advantageous in some processes, since larger crystals are easier to separate from the slurry. However larger crystals typically require longer residence times. A narrow size distribution is advantageous since a continuous separation method may be optimised to separate particles of a certain size from the slurry and a narrow size distribution ensures the maximum number are of the optimal size. Crystalline cannabinoid particles with a size that closely matches the expected size will therefore also be closest to the optimal size of the separation method.

At step 308, the crystalline cannabinoid particles are continuously separated from the cannabinoid deplete solution. Continuous separation may include centripetal separation, filter separation, and settling. As mentioned above, continuously separating crystalline cannabinoid particles is more efficient when the size and size distribution of the crystalline cannabinoid particles closely matches the size for which the separation method has been optimised. Separating the crystalline cannabinoid particles from the cannabinoid-depleted solution may include forming a mother liquor. Method 300 may further comprise reducing the volume of the mother liquor to form a concentrated mother liquor and a recycled solvent.

Method 300 may further comprise mixing the concentrated mother liquor with a second cannabinoid solution to form a follow-on quantity of cannabinoid-rich solution. The follow-on quantity of cannabinoid-rich solution may comprise at least a portion of the first cannabinoid. Mixing the concentrated mother liquor with the second cannabinoid solution to form the follow-on quantity of cannabinoid-rich solution may increase the efficiency of the crystallization, since a portion of the dissolved cannabinoid that was not initially crystallized may crystallize when the concentrated mother liquor passes through the tubular reactor.

Method 300 may further comprise dissolving at least a portion of the cannabinoid in the recycled solvent to form a third cannabinoid solution, dissolving the remainder of the cannabinoid in the solvent to form a fourth cannabinoid solution, and mixing the third and fourth cannabinoid solution to form the first cannabinoid solution. Using the recycled solvent to form the first cannabinoid solution decreases the cost of the crystallization since some of the solvent used for the crystallization may be re-used.

Flowing a first cannabinoid solution through a tubular reactor (100) under turbulent flow conditions may include flowing the first cannabinoid solution at a first flow velocity through the tubular reactor (100), wherein the tubular reactor has a first hydraulic diameter, and wherein the first flow velocity and the first hydraulic diameter combine to give the tubular reactor a Reynold's number greater than 2,900. A Reynolds number of 2,900 may be the critical Reynolds number for flow in a suitable pipe, therefore flow conditions comprising a Reynolds number greater than 2,900 comprise turbulent flow conditions in the present context. For cylindrical pipes, the hydraulic diameter is equal to the pipe diameter. For non-cylindrical pipes, the hydraulic diameter is the diameter of a cylindrical pipe with a flow rate equal to the flow rate through said non-cylindrical pipe.

In the method 300, the tubular reactor (100) comprises a plurality of baffles (120). Each of the plurality of baffles (120) possesses a shape that partially obstructs fluid flow through the tubular reactor (100). Flowing a cannabinoid-rich solution through a tubular reactor (100) under turbulent flow conditions may include flowing the cannabinoid-rich solution through the plurality of baffles (120), each of which is positioned and oriented within the tubular reactor to form a plurality of inter-baffle zones within the tubular reactor. Each inter-baffle zone may comprise a portion of the tubular reactor located between two adjacent baffles. Flowing the cannabinoid-rich solution through the tubular reactor (100) under turbulent flow conditions may include generating vortices within the plurality of inter-baffle zones due to the partially obstructed flow of the first cannabinoid solution through plurality of baffles. Inter-baffle zones may provide sufficient volume within the tubular reactor (100) in which vortices may propagate to ensure good mixing of the fluid within the tubular reactor (100). Each of the plurality of baffles (120) may comprise an orifice baffle with a circular opening (122). Generating vortices within the plurality of inter-baffle zones may include generating vortices by flowing the cannabinoid-rich solution through the circular opening (122) of the baffle (120).

Flowing a cannabinoid-rich solution through a tubular reactor (100) under turbulent flow conditions may include oscillating the flow of the cannabinoid-rich solution through the tubular reactor with an oscillation frequency and an oscillation amplitude, and the Reynolds number may comprise an oscillatory Reynolds number. The critical Reynolds number for turbulent flow may comprises a critical oscillatory Reynolds number for turbulent flow. An oscillatory Reynolds number is similar in some ways to the Reynolds number for flow in a pipe. An oscillatory Reynolds number increases with increasing amplitude and increasing frequency as set out in EQN. 1. Flow conditions with an oscillatory Reynolds number above a critical oscillatory Reynolds number for turbulent flow of about 2900 comprise turbulent flow conditions.

Oscillating the flow of the first cannabinoid-rich solution through the tubular reactor (100) may include oscillating the flow with a piston, a diaphragm pump, or oscillating the pressure applied to the inlet (112) and/or the outlet (114). For example, an oscillating the flow may be executed by reversibly translating the piston at a frequency between about 0.1 Hz and about 6.0 Hz on the laboratory scale. Those skilled in the art who have benefitted from the teachings of the present disclosure will appreciated that larger scale tubular reactors may be suited to alternate piston-oscillation protocols. Alternatively, the plurality of baffles (120) may not be physically coupled to the tubular reactor (120), but may instead be physically coupled to one another. In this case, the flow within the tubular reactor (100) may be oscillated by oscillating the position of the baffles within the tubular reactor (100). The oscillation amplitude may be greater than the net flow rate (148), which may be advantageous due to the occurrence of back flow, where back flow may increase the mixing within the tubular reactor (100).

Flowing a cannabinoid-rich solution through a tubular reactor (100) under turbulent flow conditions may include flowing the cannabinoid-rich solution through a tubular reactor surrounded by a jacket. The jacket may be thermally coupled to the tubular reactor. Method 300 may further comprise circulating a thermal fluid through the jacket. The thermal coupling between the jacket and the tubular reactor (100) allows heat to be transferred into or out of the tubular reactor (100) if the thermal fluid is warmer than or colder than the tubular reactor, respectively. Heat transfer to or from the tubular reactor (100) allows the temperature of the fluid within the tubular reactor (100) to be controlled, which is advantageous due to the strong variation of nucleation and crystallization rates with varying temperature.

The jacket may comprise a plurality of sub-jackets. The thermal fluid may comprise a plurality of portions of thermal fluid, each portion of thermal fluid possessing a respective thermal fluid portion temperature. Circulating the thermal fluid through the jacket may include circulating each of the portions of thermal fluid through a respective sub-jacket to form a temperature gradient within the tubular reactor. Since each portion of thermal fluid circulates through only one of the sub-jackets, the portion of the tubular reactor (100) thermally coupled to the sub-jacket will transfer heat to or from the respective portion of thermal fluid. By establishing a temperature gradient across the portions of thermal fluid, a temperature gradient may be established across the tubular reactor (100). The thermal gradient may comprise an initial temperature between about 0° C. and about 50° C., and a final temperature between about −10° C. and about 40° C.

In the context of method 300, the cannabinoid-rich solution may comprise a solvent, or a mixture of solvents. A mixture of solvents may be advantageous to achieve a desired nucleation or crystal growth rate. The solvent may comprise a Class III solvent, where Class III solvents are advantageous for active pharmaceutical ingredient preparation due to their low toxicity. The solvent may be heptane, as heptane has minimal toxicity and low flammability characteristics.

### Examples

### Crystallization of Cannabidiol (CBD) in a Continuous Oscillatory Baffled Crystallizers (COBC)

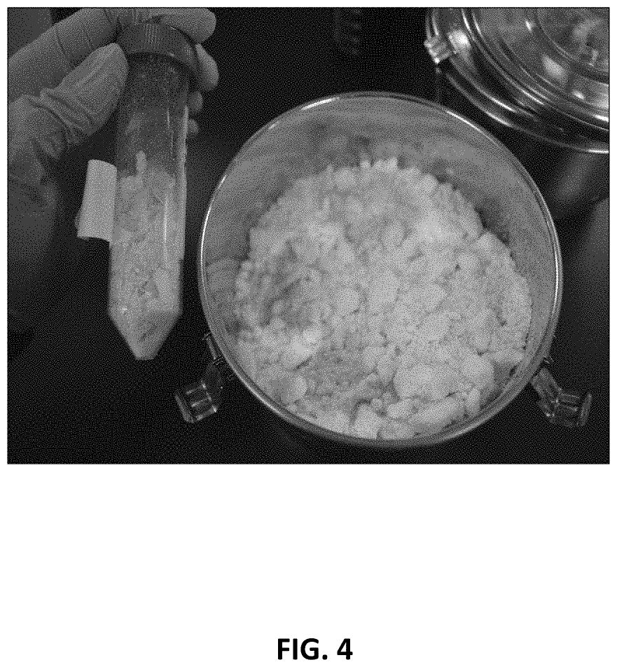

A COBC was used to prepare crystalline CBD across a series of trials with varying crystallization parameters in accordance with the methods of the present disclosure. FIG. 4 shows an exemplary harvest from one such trial. Across the series, the COBC was configured to provide oscillatory Reynolds numbers, Reₒ, between about 3,000 and about 15,000. For example, a number of trials were executed at a Reynolds number of about 6,000. Across the series, the CBD-input material was prepared as a slurry comprising a CBD solution in heptane and suspended CBD seed crystals. Across the series, the CBD solution was prepared using either CBD isolate or CBD distillate. Across the series, the suspended CBD seed crystals were prepared by conventional batch crystallization, and they were crushed and screened through a mesh filter before addition to the suspension. The concentration of the CBD solution and the quantity of CBD seed crystals was varied across the series, as was the temperature profile of the COBC. For example, COBC-inlet temperatures of between about 10° C. and about 30° C. were evaluated as were COBC-outlet temperatures between about −5° C. and about −20° C. COBC residence time, oscillation parameters (e.g. stroke length and frequency), and solvent composition were also varied across the series. The resulting crystallization yields varied across the series. For example, one trial provided a crystallization yield of about 56%, while another provided a crystallization yield of about 38%.

### Crystal Size and Morphology Comparisons

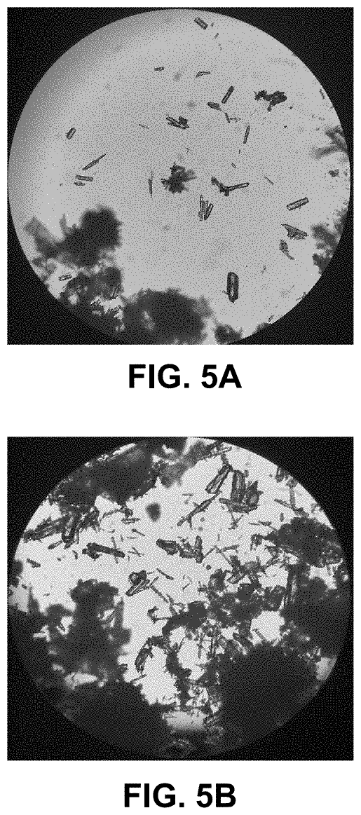

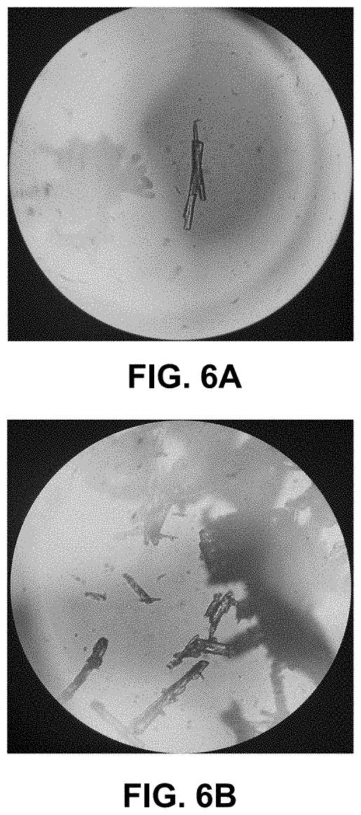

Crystals obtained from the series of trials were compared under microscope to those obtained by conventional batch processes. FIG. 5A shows a 10× magnification of crystals obtained from one trial in accordance with the present disclosure. FIG. 5B shows a 10× magnification of crystals obtained from a conventional batch process. FIG. 6A shows a 40× magnification of crystals obtained from one trial in accordance with the present disclosure. FIG. 6B shows a 40× magnification of crystals obtained from a conventional batch process. Comparison of the crystal size and morphology between: (i) FIG. 5A and FIG. 5B, and/or (ii) FIG. 6A and FIG. 6B indicate that select methods of the present disclosure provide access to crystalline cannabinoid materials with narrower size distributions, and/or narrow polymorphic profiles.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims. Moreover, although not explicitly described in the present disclosure, methods that provide all polymorphic forms and sizes of crystalline cannabinoids produced by continuous crystallization fall within the scope of the appended claims. Moreover, the crystallinity of the materials produced by the methods of the present disclosure should not be construed as limiting. For example particles produced by the methods of the present disclosure may be crystalline or amorphous.

1. A method for producing crystalline cannabinoid particles in continuous mode, the method comprising:
preparing a cannabinoid-rich solution that comprises a first cannabinoid;
inducing the cannabinoid-rich solution to a supersaturated state in which the first cannabinoid has a supersaturated concentration that is at greater than a corresponding saturation concentration of the first cannabinoid;
flowing the cannabinoid-rich solution through a tubular reactor in a continuous manner under turbulent flow conditions to form a plurality of crystalline cannabinoid particles and a cannabinoid-depleted solution within the tubular reactor and to provide a net flow rate through the tubular reactor; and
separating crystalline cannabinoid particles from the plurality of crystalline cannabinoid particles, the cannabinoid-depleted solution, or a combination thereof,
wherein the turbulent flow conditions are defined by a Reynold number that is greater than a critical Reynolds number for the cannabinoid-rich solution and the tubular reactor.

2. The method of claim 1, wherein the critical Reynolds number greater than 2,300.

3. The method of claim 1, wherein the critical Reynolds number greater than 2,900.

4. The method of claim 1, wherein the critical Reynolds number greater than 3,900.

5. The method of claim 1, wherein the Reynolds number is about 6,000.

6. The method of claim 1, wherein the net flow rate is between about 10 mL/min and about 100 mL/min.

7. The method of claim 1, further comprising superimposing an oscillating flow rate on the net flow rate by oscillating a piston that is in fluid communication with the tubular reactor.

8. The method of claim 1, wherein the tubular reactor comprises a baffle that is shaped, oriented, or positioned to partially obstruct flow through the tubular reactor.

9. The method of claim 8, wherein the baffle is one of a plurality of baffles.

10. The method of claim 8, further comprising oscillating the baffle within the tubular reactor to superimpose an oscillating flow rate on top of the net flow rate.

11. The method of claim 1, further comprising cooling the cannabinoid-rich solution, the cannabinoid-depleted solution, or a combination thereof within the tubular reactor.

12. The method of claim 1, wherein the first cannabinoid is THC (Δ9-THC), THCA, Δ8-THC, trans-Δ10-THC, cis-Δ10-THC, THCV, THCVA, Δ8-THCV, Δ9-THCV, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA CBL, CBLA, CBLV, CBLVA, CBT, CBTA, or cannabicitran.

13. The method of claim 1, wherein the cannabinoid-rich solution comprises a cannabinoid extract, a cannabinoid resin, a cannabinoid distillate, a cannabinoid isolate, or a combination thereof.

14. The method of claim 1, wherein the cannabinoid-rich solution comprises THC (Δ9-THC), THCA, Δ8-THC, trans-Δ10-THC, cis-Δ10-THC, THCV, THCVA, Δ8-THCV, Δ9-THCV, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA CBL, CBLA, CBLV, CBLVA, CBT, CBTA, cannabicitran, or a combination thereof.

15. The method of claim 1, wherein the cannabinoid-rich solution comprises a solvent, and wherein the solvent comprises pentane, hexane, heptane, methanol, ethanol, isopropanol, dimethyl sulfoxide, acetone, ethyl acetate, diethyl ether, tert-butyl methyl ether, water, acetic acid, anisole, 1-butanol, 2-butanol, butane, butyl acetate, ethyl formate, formic acid, isobutyl acetate, isopropyl acetate, methyl acetate, 3-methyl-1-butanol, methylethyl ketone, 2-methyl-1-propanol, 1-pentanol, 1-propanol, propane, propyl acetate, trimethylamine, or a combination thereof.

16. The method of claim 1, wherein the cannabinoid-rich solution has a viscosity of between about 0.05 cP and about 250 cP at an inlet to the tubular reactor.

17. The method of claim 1, wherein the cannabinoid-rich solution has a fluid density of between about 0.2 g/mL and about 1,700 g/mL at an inlet to the tubular reactor.

18. The method of claim 1, wherein the cannabinoid-rich solution has a temperature of between about 0° C. and about 50° C. at an inlet to the tubular reactor.

19. The method of claim 1, wherein the inducing of the cannabinoid-rich solution to the supersaturated state precedes the flowing of the cannabinoid-rich solution through the tubular reactor.

20. The method of claim 1, wherein the inducing of the cannabinoid-rich solution to the supersaturated state is concurrent the flowing of the cannabinoid-rich solution through the tubular reactor.

21. The method of claim 1, wherein the cannabinoid-rich solution further comprises and excipient.

22. The method of claim 1, further comprising dispersing a plurality of seed crystals into the cannabinoid-rich solution concurrent with the flowing of the cannabinoid-rich solution through the tubular reactor.
